(19) United States
(12) Patent Application Publication
(10) Pub. No.: US 2025/0121155 A1
(43) Pub. Date: Apr. 17, 2025

(54) SYSTEM FOR PROVIDING COGNITIVE FUNCTION ENHANCEMENT SERVICE USING ARTIFICIAL INTELLIGENCE-BASED BRAINWAVE ENTRAINMENT AND LIGHT THERAPY

(71) Applicants: JangWon LEE, Seoul (KR); Meta Therapeutics Inc., Gwangju (KR)

(72) Inventor: JangWon LEE, Seoul (KR)

(73) Assignees: JangWon LEE, Seoul (KR); Meta Therapeutics Inc., Gwangju (KR)

(21) Appl. No.: 18/796,409

(22) Filed: Aug. 7, 2024

(30) Foreign Application Priority Data
Oct. 17, 2023 (KR) 10-2023-0138609

(51) Int. Cl. A61M 21/02; A61M 21/00; G16H 20/30

(52) U.S. Cl. CPC A61M 21/02; G16H 20/30; A61M 2021/0027; A61M 2021/005; A61M 2021/0083; A61M 2205/18; A61M 2205/3327; A61M 2205/3553; A61M 2205/502

(57) ABSTRACT

Disclosed is a system for providing a cognitive function enhancement service using AI-based brainwave entrainment and light therapy, the system including: a user terminal configured to output blue light for cognitive function enhancement, insert and output a beta-band or gamma-band flicker for brainwave entrainment of beta waves or gamma waves to enhance concentration and memory, output monaural or binaural beats in an inaudible band for brainwave entrainment to enhance concentration and memory, and collect cognitive function measurement results; and a server for providing a cognitive function enhancement service.

### BACKGROUND OF THE INVENTION

The present disclosure relates to a system for providing a cognitive function enhancement service using AI-based brainwave entrainment and light therapy and, more specifically, to a method for utilizing brainwave entrainment and light therapy to enhance a cognitive function, measuring results, and providing the measurement results.

Sleep is one of the biological rhythms of the body, and the 24-hour Circadian rhythm is regulated by an internal clock located in the suprachiasmatic nucleus of the brain and also by external factors known as Zeitgebers. Destruction of the suprachiasmatic nucleus eliminates the Circadian rhythm even if sleep cycles persist. Studies suggest that the actual sleep requirement for adults is approximately 8.5 hours when allowed to follow natural sleep patterns, but more sleep is generally needed during physical labor, exercise, illness, pregnancy, mental stress, and mental activities. Decreased sleep can impair cognitive function, particularly affecting functions associated with the prefrontal cortex and cortical areas. Deficits in alertness and delayed reaction times are prominently observed, exacerbated in the morning due to circadian influences. Consequently, inadequate sleep may lead to fatigue and lethargy in daily life, while severe deficiencies could predispose individuals to chronic fatigue, dementia, depression, and anxiety disorders.

In this context, methods have been researched and developed to enhance cognitive function using brainwave entrainment and aid sleep disorders by mixing monaural beats. Related technologies are disclosed in Korean Patent No. 10-2113547 (published on May 21, 2020) and Korean Patent Application No. 2023-0080260 (published on Jun. 7, 2023), detailing configurations that stimulate the brain and synchronize brainwaves by utilizing frequencies matching the user's unique frequencies during cognitive task performance, mixing waveform types of monaural beats to induce brainwave entrainment, loading frequency data according to standard frequencies for brainwave entrainment, adjusting decibels, and delivering overlaid monaural beats as waveform files to user terminals.

However, the former only discloses configurations providing frequencies identical to the user's unique frequencies, and the latter only discloses configurations using light therapy and sound therapy, without mentioning monaural or binaural beats or specifying the types of light therapy. Brainwaves and the autonomic nervous system are regulated according to sleep and wakefulness, and insomnia or hypersomnia tends to indicate deviations from normal ranges in brainwave and autonomic nervous system regulation. Moreover, abnormal activation in the hypothalamus, which is responsible for circadian rhythms, can disrupt these rhythms and cause cluster headache, as well as various symptoms such as depression, ADHD, anxiety disorders, obsessive-compulsive disorder, panic disorder, and cognitive impairments. Therefore, research and development of systems that induce deep sleep to manage sleep cycles and ultimately enhance cognitive function are necessary.

### SUMMARY OF THE INVENTION

In view of the above, the present disclosure provides a system for providing a cognitive function enhancement service using AI-based brainwave entrainment and light therapy, the system which enhances a cognitive function using brainwave entrainment and light therapy, enhances a cognitive function by inducing deep sleep with monaural or binaural beats, manages sleep cycles using light and sound-based wake-up alarms, induces brainwave entrainment using light and sound to improve symptoms of mild cognitive impairment, Alzheimer's disease, and ADHD, collects bio-data to provide feedback when receiving improvement processes using machine learning, and identifies correlations and effects. However, the technical challenge that this embodiment aims to achieve is not limited to the technical challenges described above, and other technical challenges may exist.

In one general aspect, there is provided a system for providing a cognitive function enhancement service using AI-based brainwave entrainment and light therapy, and the system includes: a user terminal configured to output blue light for cognitive function enhancement, insert and output a beta-band or gamma-band flicker for brainwave entrainment of beta waves or gamma waves to enhance concentration and memory, output monaural or binaural beats in an inaudible band for brainwave entrainment to enhance concentration and memory, and collect cognitive function measurement results; and a server for providing a cognitive function enhancement service, which comprises a controller configured to control the blue light, light of a beta band or gamma band, and sound of monaural or binaural beats, a collector configured to collect the cognitive function measurement results from the user terminal, an extractor configured to extract a factor that correlates the blue light, the light of the beta band or gamma band, and the sound of the monaural or binaural beats with the cognitive function measurement results by using pre-established machine learning, and an application part configured to provide the extracted factor to the user terminal for application.

According to the present disclosure, it is possible to enhance cognitive function through brainwave entrainment and light therapy; to enhance a cognitive function by inducing deep sleep with monaural or binaural beats; to manage sleep cycles using light and sound-based wake-up alarms; to induce brainwave entrainment using light and sound to improve symptoms of mild cognitive impairment, Alzheimer's disease, and ADHD; to collect bio-data to provide feedback when receiving improvement processes using machine learning; and to identify correlations and effects.

### BRIEF DESCRIPTION OF THE DRAWINGS

FIG. 1 is a diagram illustrating a system for providing a cognitive function enhancement service using AI-based brainwave entrainment and light therapy according to an embodiment of the present disclosure.

FIG. 2 is a block diagram illustrating an improved service providing server included in the system of FIG. 1.

FIGS. 3A-3B and 4A-4F are diagrams for explaining examples of a cognitive function enhancement service using AI-based brainwave entrainment and light therapy according to an embodiment of the present disclosure.

FIG. 5 is an operation flowchart illustrating a method for providing a cognitive function enhancement service using AI-based brainwave entrainment and light therapy according to an embodiment of the present disclosure.

### DETAILED DESCRIPTION OF THE INVENTION

Hereinafter, exemplary embodiments of the present disclosure will be described in detail with reference to the accompanying drawings. The present disclosure, however, may be modified in various different ways, and should not be construed as limited to the embodiments set forth herein. For clarity of the disclosure, irrelevant parts are removed from the drawings, and similar reference denotations are used to refer to similar elements throughout the specification.

In embodiments of the present disclosure, when an element is “connected” with another element, the element may be “directly connected” with the other element, or the element may be “electrically connected” with the other element via an intervening element. When an element “comprises” or “includes” another element, the element may further include, rather than exclude, the other element, and the terms “comprise” and “include” should be appreciated as not excluding the possibility of presence or adding one or more features, numbers, steps, operations, elements, parts, or combinations thereof.

When the measurement of an element is modified by the term “about” or “substantially,” if a production or material tolerance is provided for the element, the term “about” or “substantially” is used to indicate that the element has the same or a close value to the measurement and is used for a better understanding of the present disclosure or for preventing any unscrupulous infringement of the disclosure where the exact or absolute numbers are mentioned. As used herein, “step of” A or “step A-ing” does not necessarily mean that the step is one for A.

As used herein, the term “part” may mean a unit or device implemented in hardware, software, or a combination thereof. One unit may be implemented with two or more hardware devices or components, or two or more units may be implemented in a single hardware device or component. The “unit” is not limited to software or hardware devices or components and may advantageously be configured to reside on an addressable storage medium and configured to operate on one or more processors. Accordingly, the “unit” may include, for example, components, such as software components, object-oriented software components, class components, and task components, processes, functions, attributes, procedures, sub-routines, segments of program code, drivers, firmware, microcode, circuitry, data, databases, data structures, tables, arrays, and variables. The functionalities provided in the components and “units” may be combined into fewer components and “units” or may be further separated into additional components and “units.” Furthermore, the components and “units” may be implemented to operate on one or more CPUs within a device or a security multimedia card.

In the present specification, some of the operations or functions described as performed by a terminal, an apparatus, or a device may be performed instead in a server connected to the corresponding terminal, apparatus, or device. Similarly, some of the operations or functions described as being performed by a server may be performed in a terminal, an apparatus, or a device connected to the corresponding server.

In the present specification, some of the operations or functions described as mapping with or matching a terminal means mapping or matching a unique number of the terminal or personal identification information, which is identification data of the terminal.

Hereinafter, the present disclosure will be described in detail with reference to the attached drawings.

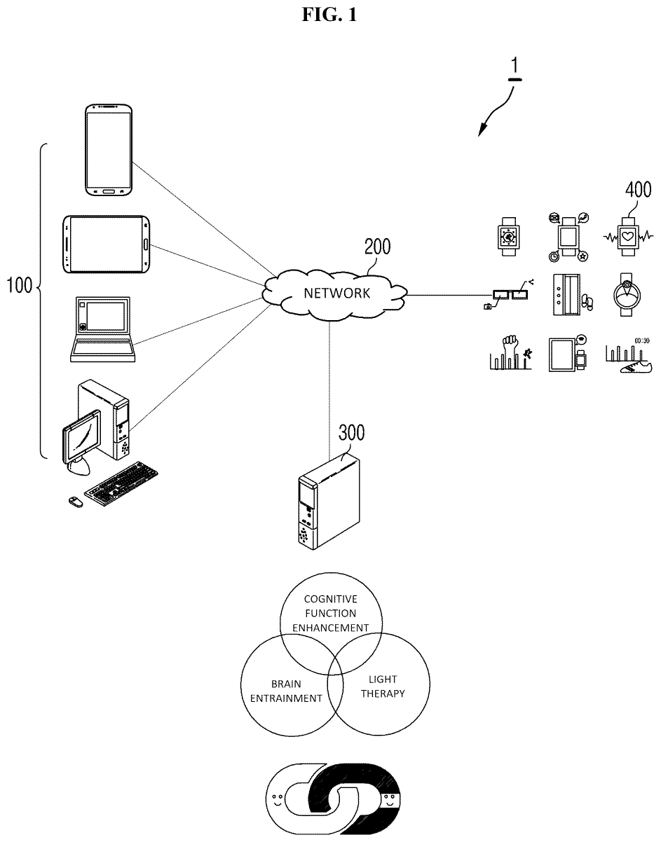

FIG. 1 is a diagram illustrating a system for providing a cognitive function enhancement service using AI-based brainwave entrainment and light therapy according to an embodiment of the present disclosure. Referring to FIG. 1, a system 1 for providing a cognitive function enhancement service using AI-based brainwave entrainment and light therapy may include at least one user terminal 100, a server 300 for cognitive function enhancement, and at least one wearable device 400. However, since the system 1 shown in FIG. 1 is merely an example of the present disclosure, the present disclosure is not limited thereby.

In this case, each component of FIG. 1 is generally connected over a network 200. For example, as shown in FIG. 1, at least one user terminal 100 may be connected to the sleep healthcare server 300 over the network 200. In addition, the sleep healthcare server 300 may be connected to at least one user terminal 100 and at least one wearable device 400 over the network 200. Moreover, at least one wearable device 400 may be connected to the sleep healthcare server 300 over the network 200.

Here, the network refers to a connected structure that enables information exchange between nodes such as multiple terminals and servers. Examples of such a network include a Local Area Network (LAN), Wide Area Network (WAN), Internet (World Wide Web, WWW), wired and wireless data communication networks, telephone networks, and wired and wireless television communication networks. Examples of wireless data communication networks include 3G, 4G, 5G, 3rd Generation Partnership Project (3GPP), 5th Generation Partnership Project (5GPP), 5G New Radio (NR), 6th Generation of Cellular Networks (6G), Long Term Evolution (LTE), World Interoperability for Microwave Access (WiMAX), Wi-Fi, Internet, LAN, Wireless Local Area Network (Wireless LAN), WAN, Personal Area Network (PAN),

Radio Frequency (RF), Bluetooth networks, Near-Field Communication (NFC) networks, satellite broadcast networks, analog broadcast networks, and Digital Multimedia Broadcasting (DMB) networks, but are not limited thereto.

In the following, the term “at least one” is defined as a term including the singular and plural, and even if the term “at least one” is not present, each component may be present in singular or plural, and it will be obvious that it may mean singular or plural. In addition, whether each component is provided in singular or plural forms can vary according to embodiments.

At least one user terminal 100 may be a device that outputs light or sound for brainwave entrainment and emits blue light for light therapy using a web page, app page, program, or application related to a cognitive function enhancement service. In addition, the user terminal 100 may be a device that inputs a response to an attention task and outputs feedback-controlled light, sound, and blue light accordingly.

Here, at least one user terminal 100 may be implemented as a computer capable of accessing a server or terminal at a remote location over a network. The computer may include, for example, a navigation system, a laptop, a desktop, and a laptop equipped with a web browser (WEB Browser), etc. At least one user terminal 100 may be implemented as a terminal capable of accessing a server or terminal at a remote location over a network. At least one user terminal 100 may be a mobile communication device in which portability and mobility are guaranteed, and examples thereof may include all types of handheld-based wireless communication devices such as a personal communication system (PCS), global system for mobile communication (GSM), personal digital cellular (PDC), a personal handyphone system (PHS), a personal digital assistant (PDA), international mobile telecommunication (IMT)-2000, code division multiple access (CDMA)-2000, W-code division multiple access (W-CDMA), a wireless broadband Internet (WiBro) terminal, a smart phone, a smart pad, a tablet PC, and the like.

The server 300 may be a server that provides the web page, app page, program, or application for a cognitive function enhancement service using AI-based brainwave entrainment and light therapy. In addition, the server 300 may be a server that stores, in a database, control data for light, sound, and blue light for cognitive function enhancement. Furthermore, the server 300 may be a server that checks whether cognitive function has been improved after the output of light, sound, and blue light from the user terminal 100, measures the results, extracts significant factors using machine learning, and identifies frequencies, cycles, and intensities of the light, sound, and blue light. In addition, the server 300 may be a server that provides light, sound, and blue light with adjusted frequency, periodicity, intensity, etc.

Here, the sleep healthcare server 300 may be implemented as a computer capable of accessing a server or terminal at a remote location over a network. The computer may include, for example, a navigation system, a laptop, a desktop, and a laptop equipped with a web browser (WEB Browser), etc.

At least one wearable device 400 may be a device that acquires biological data by utilizing a web page, app page, program, or application related to the cognitive function enhancement service using AI-based brainwave entrainment and light therapy.

Here, at least one wearable device 400 may be implemented as a computer capable of accessing a server or terminal at a remote location over a network. The computer may include, for example, a navigation system, a laptop, a desktop, and a laptop equipped with a web browser (WEB Browser), etc. In this case, at least one wearable device 400 may be implemented as a terminal capable of accessing a server or terminal at a remote location over a network. At least one wearable device 400 may be a mobile communication device in which portability and mobility are guaranteed, and examples thereof may include all types of handheld-based wireless communication devices such as a personal communication system (PCS), global system for mobile communication (GSM), personal digital cellular (PDC), a personal handyphone system (PHS), a personal digital assistant (PDA), international mobile telecommunication (IMT)-2000, code division multiple access (CDMA)-2000, W-code division multiple access (W-CDMA), a wireless broadband Internet (WiBro) terminal, a smart phone, a smart pad, a tablet PC, and the like.

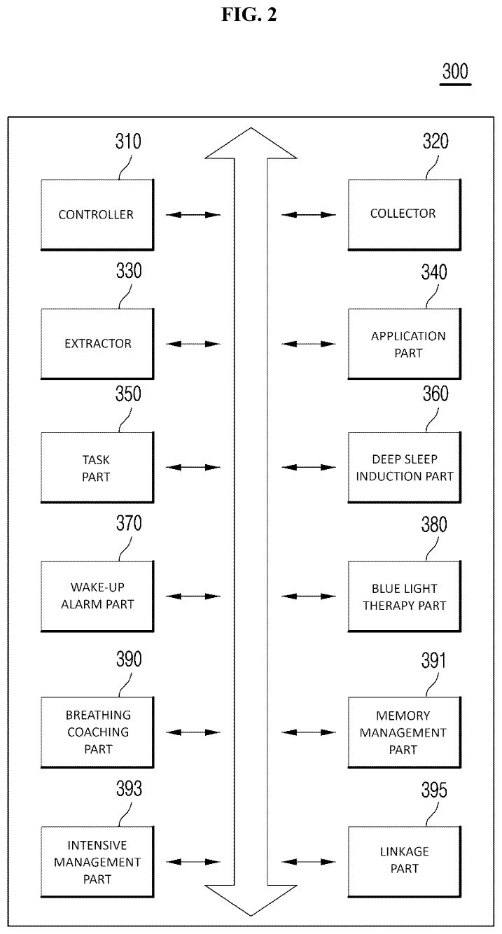

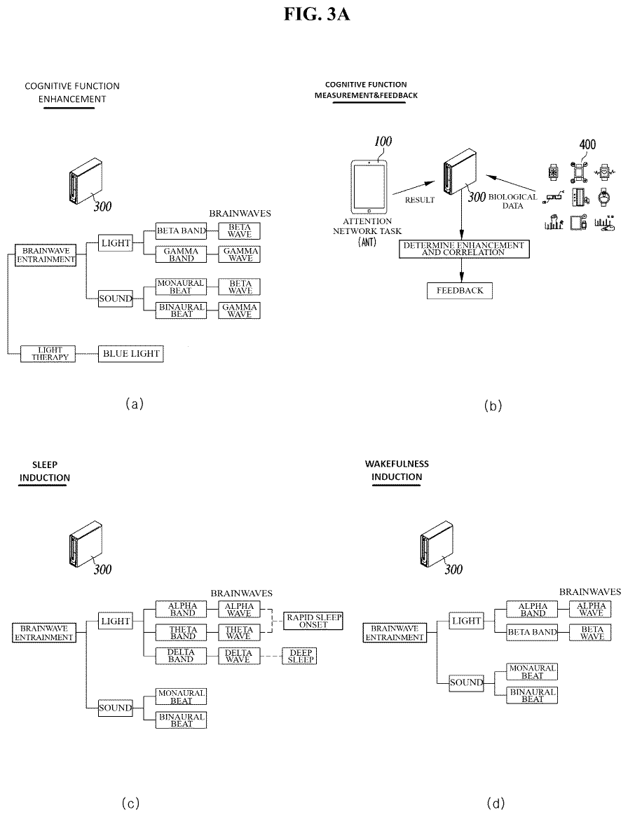

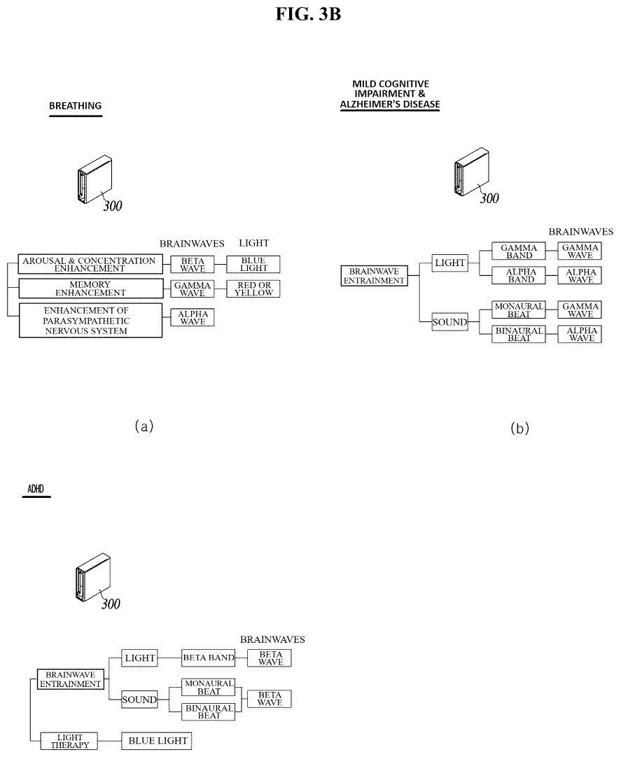

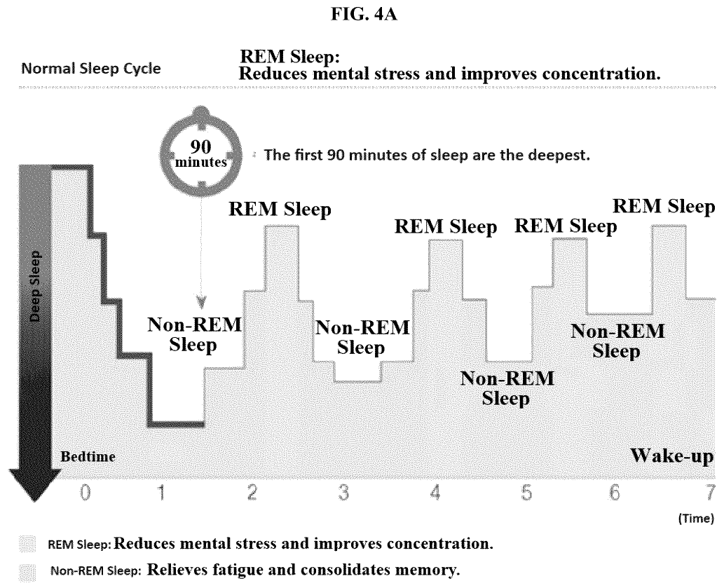

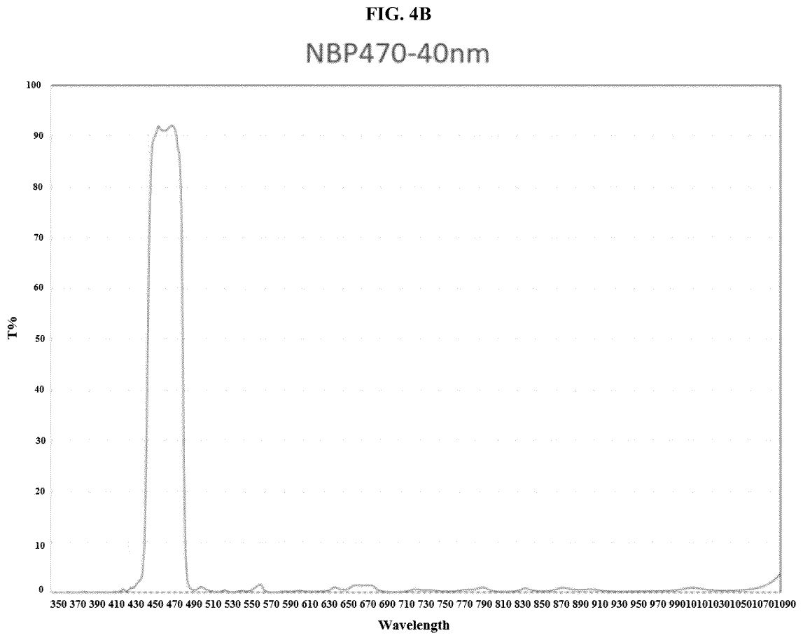

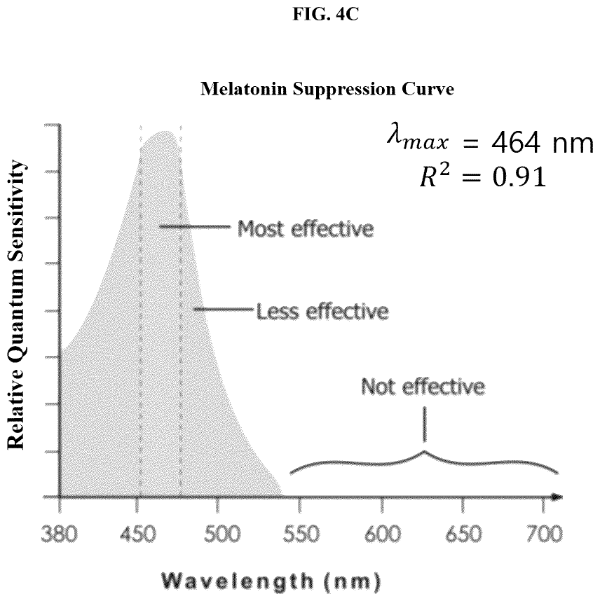

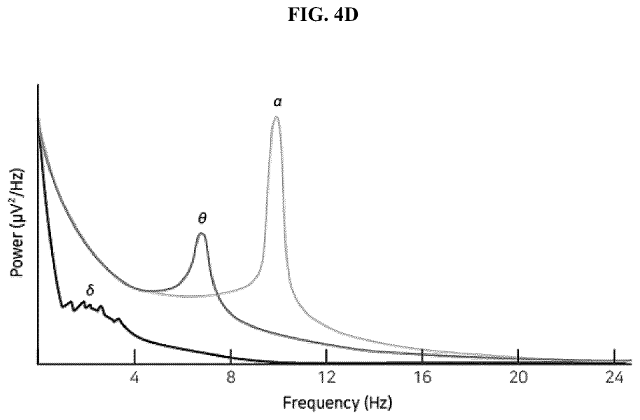

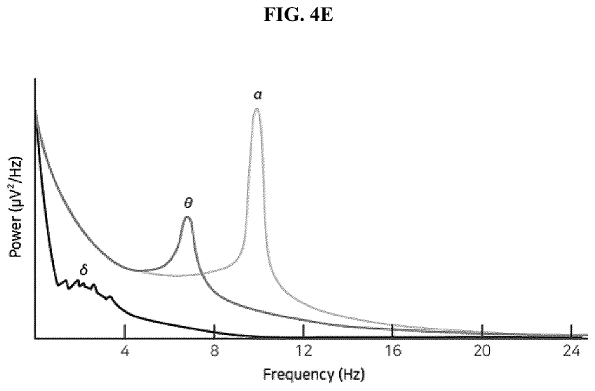

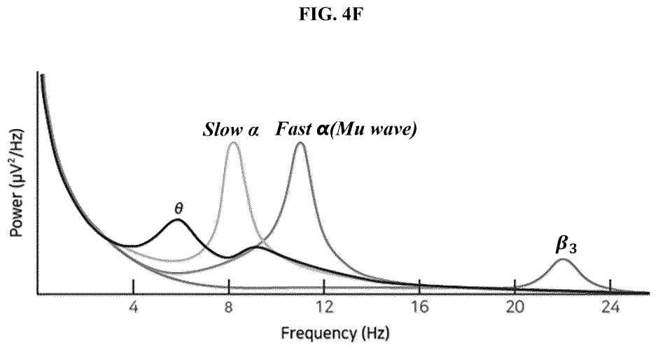

FIG. 2 is a block diagram for explaining the service for cognitive function enhancement, included in the system of FIG. 1, and FIGS. 3 and 4 are diagrams for explaining examples in which a cognitive function enhancement service using AI-based brainwave entrainment and light therapy according to an embodiment of the present disclosure is implemented.

Referring to FIG. 2, the server 300 may include a controller 310, a collector 320, an extractor 330, an application part 340, a task part 350, and a deep sleep induction part 360, a wake-up alarm part 370, a blue light therapy part 380, a breathing method coaching part 390, a memory management part 391, an intensive management part 393, and a linkage part 395.

According to an embodiment of the present disclosure, when the server 300 or another server (not shown) operatively connected thereto transmits an application, program, app page, web page, or the like for a cognitive improvement service using AI-based brainwave entrainment and light therapy to at least one user terminal 100 and at least one wearable device 400, the at least one user terminal 100 and the at least one wearable device 400 may install or open the application, program, app page, web page, or the like for a cognitive improvement service using AI-based brainwave entrainment and light therapy. In addition, the service program may be executed on the at least one user terminal 100 and the at least one wearable device 400 using scripts executed in a web browser. Here, the web browser is a program that enables the use of web (WWW: World Wide Web) services and displays hypertext described in HTML (Hyper Text Mark-up Language), and examples of the web browser include Chrome, Microsoft Edge, Safari, Firefox, Whale, UC Browser, and the like. Additionally, an application refers to an application on a terminal, and for example, includes an app able to be executed on a mobile terminal (smartphone).

Referring to FIG. 2, the controller 310 may control light such as blue light, light of beta band or gamma band, and sound of monaural beats or binaural beats. In one embodiment of the present disclosure, light and sound are used for brainwave entrainment, while light therapy utilizes blue light or light other than blue light. This is summarized in Table 1 below.

TABLE 1

| | | |
|---|---|---|
| Brainwave Entrainment | Light | Alpha Band |
| | | Beta Band |
| | | Gamma Band |
| | | Theta Band |
| | | Delta Band |
| | Sound | Monaural Beats |
| | | Binaural Beats |
| Light Therapy | Blue Light | — |
| | Light other than blue light | — |

In this case, the blue light may be generated by at least one of an LED device, a bandpass filter, a flashlight, and a display. The light for beta bands or gamma bands may be controlled to create flicker by inserting image frames with flickering effects into video content, adjusting a display refresh rate, or flashing at least one of a display, a flashlight, or an LED device. Here, it should be noted that all of the configurations described above may be applied to output the light in Table 1, not limited to the light for the beta or gamma bands. Therefore, repeated configurations are not listed below. Brainwaves are biometric information measured through reinforcement interference in the microcurrents of nerve cells when the brain is enhanced. Continuous data is measured at a location with amplitude, frequency, and waveform, which are characteristic of waves. Brainwaves may be used to measure brain activity because the brainwaves exhibit different characteristics depending on the activation state of neural cells. This is why polysomnography uses brainwaves to assess the depth of sleep. During sleep, a person's unconscious reduction in physical activity results in sleep brainwaves exhibiting different characteristics compared to brainwaves measured during wakefulness (non-sleep). To distinguish sleep stages and determine sleep depth using the characteristics of sleep brainwaves, the sleep stages defined in the Rechtschaffen & Kales Sleep Scoring Manual are employed. These sleep stage criteria are widely used across various research fields, including polysomnography, as shown in Table 1 below.

TABLE 2

| Stage | Frequency Characteristics | Other Characteristics |
|---|---|---|
| Wake | Alpha waves (8-13 Hz) | Noise due to movement and heart |
| | dominant | rate |
| 1 | Mixed waves (2-7 Hz) | No K-complex and sleep spindle |
| | dominant, alpha | waves |
| | waves <50% | |
| 2 | Slow waves (<2 Hz) <20% | K-complex and sleep spindle |
| | | waves detected |
| 3 | Slow waves 20-50% detected | — |
| 4 | Slow waves >50% detected | — |
| REM | Mixed waves dominant | Noise due to eye movement |

The wake stage is a non-sleep state with alpha waves (8-12 Hz) activated and noise generated due to movement and heartbeat. In stage 1 sleep, mixed brainwaves of 2-7 Hz are measured, and no K-complex and sleep spindle waves are detected. The K-complex refers to a transient, high-amplitude, positively directed waveform, while a sleep spindle is a 12-14 Hz complex wave measured shortly after the K-complex. In Stage 2 sleep, K-complexes and sleep spindles are measured, with slow waveforms below 2 Hz comprising less than 20%. Stage 3 sleep is characterized by slow brainwaves below 2 Hz, measuring between 20% and 50%. Stage 4 sleep includes more than 50% slow brainwaves below 2 Hz. REM sleep is similar to stage 1 sleep but includes noise due to eye movements. Here, the types and frequencies of brainwaves 10 are detailed in Table 3 below.

TABLE 3

| Type of | Frequency | | |
|---|---|---|---|
| Brainwaves | Band | | State of the Brain |
| Delta | 0.5~4 | Hz | Deep sleep state |
| Theta | 4~7 | Hz | Drowsiness, daydreaming, restlessness |
| Alpha | 8~12 | Hz | Relaxed with decreased external focus |
| Sensory Motor | 12~15 | Hz | Maintaining attention in stillness |
| Rhythm (SMR) | | | intermediate between tension and |
| | | | relaxation< |
| Beta | 15~30 | Hz | Active thinking and maintaining focus |
| | | | in an active state |
| Gamma | 31~50 | Hz | Information exchange between cortical |
| | | | and subcortical areas, |
| | | | Present in conscious wakefulness and |
| | | | REM sleep, |
| | | | Occasionally overlaps with beta waves |

### Brainwave Entrainment

The theory of brainwave entrainment, also known as the Frequency-Following Effect, refers to neural synchronization based on brainwave entrainment through rhythmic external stimuli such as flickering lights, voice, music, and periodic stimuli like music. According to this theory, brainwaves (the brain's large electrical oscillations) synchronize to the desired frequencies induced by these stimuli. This concept has been discussed in publications such as V. A. Korshunov, G. R. Khazankin, and D. S. Ivanishkin, “Development of an Application for Audio-Visual-Tactile Brainwave Entrainment in Patients with Affective and Psychosomatic Disorders,” 2021 IEEE 22nd International Conference of Young Professionals in Electron Devices and Materials (EDM), Souzga, the Altai Republic, Russia, 2021, pp. 551-554, doi: 10.1109/EDM52169.2021.9507617, and H. Norhazman, N. Zaini, M. N. Taib, R. Jailani, and M. F. A. Latip, “Alpha and Beta Sub-waves Patterns when Evoked by External Stressor and Entrained by Binaural Beats Tone,” 2019 IEEE 7th Conference on Systems, Process and Control (ICSPC), Melaka, Malaysia, 2019, pp. 112-117, doi: 10.1109/ICSPC47137.2019.9068008. In one embodiment of the present disclosure, based on the brainwave entrainment theory, light (alpha band and beta band) and sound (monaural beats and binaural beats) are utilized to induce sleep and promote arousal using blue light. In one embodiment of the present disclosure, based on the brainwave entrainment theory, a cognitive function may be improved using light (beta and gamma bands), sound (monaural beats and binaural beats), and blue light.

### Blue Light for Cognitive Function Enhancement

The theoretical basis for using blue light to enhance cognitive function is based on a study (Lin Z, Hou G, Yao Y, Zhou Z, Zhu F, Liu L, Zeng L, Yang Y, Ma J. 40-Hz Blue Light Changes Hippocampal Activation and Functional Connectivity Underlying Recognition Memory. Front Hum Neurosci. 2021 Dec. 16;15:739333. doi: 10.3389/fnhum.2021.739333. PMID: 34975431; PMCID: PMC8716555.). The paper examines the effects of 40 Hz and 0 Hz light exposures on regulating brain activity patterns, and reveals that only 40 Hz light enhances hippocampal activity and alters functional connectivity with other brain regions. Clear activity patterns for 40 Hz and 0 Hz stimuli indicate different response patterns of brain regions distributed in a memory-related brain network. These findings are consistent with the concept that memory discernment is confined to the hippocampus, and further predict the optimizing effect of gamma light stimulation on brain functions related to memory. These data suggest that using 40 Hz frequency light can indeed alter hippocampal activity and brain functional connectivity related to memory tasks.

The collector 320 may collect cognitive function measurement results from the user terminal 100. The user terminal 100 may output blue light for cognitive function enhancement, insert flickers of beta-band (Beta-Band) or gamma-band (Gamma-Band) for brainwave entrainment aimed at improving concentration and memory, output monaural beats or binaural beats in an inaudible band for brainwave entrainment aimed at improving concentration and memory, and collect cognitive measurement results.

By using pre-established machine learning, the extractor 330 may extract a factor that has correlations between blue light, light of beta-band or gamma-band, sound of monaural beats or binaural beats, and cognitive measurement results. In other words, the correlation of [blue light-light-sound-cognitive function] may be identified. In this case, independent and dependent variables are set. In one embodiment of the present disclosure, two methods may be used to determine which variables are relatively important among the various variables (factors). One method is to derive variable importance to identify which variables are significant among the various variables, while the other method is to improve the limitations of black-box models by using eXplainable Artificial Intelligence (XAI) to explain the basis of AI's decisions when analyzing the correlation between each variable and a corresponding result thereof through correlation analysis.

### Deriving Important Variables

Recently, as various studies on prediction and significant predictors have been conducted, machine learning methods have been widely used. Machine learning methods include models whose results are interpretable, and black-box models whose results are interpretable. While it is essential to use methods with poor model interpretability but good prediction accuracy to predict the regression and classification of response variables, it is also crucial to go beyond building a predictive model to explore and identify highly correlated explanatory variables. To this end, it is necessary for machine learning techniques used for prediction and classification to have not just high prediction accuracy but also good model interpretability. Generally, as prediction accuracy increases, model interpretability decreases, a trade-off between prediction accuracy and model interpretability is necessary when building a predictive model.

In this regard, according to an embodiment of the present disclosure, decision tree-based ensemble models may be used to derive prediction accuracy and variable importance, indicating which variables are relatively crucial among the various explanatory variables. The classification of decision tree-based ensemble learning refers to a technique that generates multiple classifiers and combines the results thereof to derive a more accurate final prediction. Ensemble learning performs better than a single classifier. Table 4 below summarizes the characteristics of decision tree-based classifiers.

TABLE 4

| Model | Characteristic |
|---|---|
| Gradient Boosting | An algorithm belonging to the boosting family of |
| | ensemble methodologies |
| Light Gradient | A tree-based learning algorithm that uses Leaf- |
| Boosting | Wise tree splitting |
| Random Forest | An algorithm that aggregates classification or |
| | prediction from multiple decision trees |
| Extra Trees | An algorithm that randomly splits each candidate |
| | feature in decision trees |
| Decision Tree | An algorithm that performs regression or |
| | classification through a series of classification rules |
| Ada Boost | An algorithm where weak classifiers sequentially |
| | learn to complement each other |

Here, Leaf-Wise means continuously splitting leaf nodes without balancing the tree, resulting in an asymmetric tree, and a weak classifier (weak learner) refers to each classifier that constitutes boosting. To evaluate the superiority of models, five performance indicators may be used, such as accuracy, F1-score, precision, recall, and AUC (Area Under the Curve). Among the performance indicators, precision, recall, accuracy, and F1-score may be obtained using a confusion matrix. The confusion matrix is a cross-tabulation of predicted categories derived from a predictive model and actual categories. The elements of the confusion matrix are TP (True Positive), FP (False Positive), FN (False Negative), and TN (True Negative). TP indicates a case where both the predicted and actual values are positive, and FP indicates a case where the predicted value is positive but the actual value is negative. FN indicates a case where the predicted value is negative but the actual value is positive, and TN indicates a case where both the predicted and actual values are negative. Precision is an indicator of how accurately the model's prediction value is predicted. Recall indicates the proportion of actual positive values that the model predicted as positive. Accuracy is an indicator of how accurately predictions are made for all values. The F1-score refers to the harmonic mean of precision and recall. The AUC (Area Under ROC Curve) represents the model's accuracy and may be calculated using the area under an ROC curve. The closer all performance indicators are to 1, the better the model is. One of the key characteristics of decision trees among machine learning methods is the ability to calculate variable importance of explanatory variables during a model's learning process. The calculated variable importance may serve as a basis for understanding which input variables determine the predictive value of a decision tree, and may also be used as a basis for selecting variables in other predictive models. To calculate variable importance, the Mean Decrease in Gini Index may be used, and this index helps diagram which explanatory variables have the most explanatory power in the model. The higher the Gini index used in the decision tree, the greater the impurity of the classification. Therefore, the classification aims to reduce the Gini index.

### Correlation Analysis

As mentioned above, machine learning models may achieve high levels of prediction accuracy but often lack the ability to explain the correlation between independent and dependent variables. Here, the eXplainable Artificial Intelligence (XAI) may improve the limitations of black-box models by providing explanations for AI decisions. Therefore, it is possible to utilize a model that not only analyzes the correlation between independent and dependent variables but also explains the correlation thereof.

### XAI Algorithm

XAI algorithms such as Logistic Regression, XGBoost Classifier, and SHAP (SHapley Additive explanations) may be utilized to explain the correlation between independent and dependent variables. Logistic Regression expresses the relationship between binary data and input data in the form of an equation, and predicts the likelihood of an event occurrence. The model's form is represented by Equation 1 below.

$$log ( \frac{p}{1 - p} ) = a + {b}_{1} {x}_{1} + {b}_{2} {x}_{2} + … + {b}_{n} {x}_{n} \qquad [ Equation 1 ]$$

XGBoost combines (boosts) multiple decision trees through boosting to provide a more accurate predictive model compared to simple classifiers. XGBoost benefits from visualizing the effects of input variables at each decision step, thereby explaining correlations. SHAP uses a Shapley value to quantitatively express the contribution of each variable, and enables the calculation of the contribution while taking into account correlations between variables. In one embodiment of the present disclosure, the contribution of input variables in the XGBoost model may be quantified using Shapley values. Logistic regression, XGBoost, and SHAP can be used to perform correlation analysis. At this point, a positive correlation indicates a high correlation between the feature values of independent variable and the feature values of dependent variables. Conversely, a negative correlation indicates a low correlation between the feature values of independent variables and the feature values of dependent variables. Of course, it is evident that correlation analysis can be conducted in various other ways.

The application part 340 may provide a factor to the user terminal 100 as feedback for application.

The task part 350 may provide an attention network task based on Attention Networks Theory to obtain a cognitive measurement result. The attention network task is also called an attentional control test, which is a task that measure three attentional networks that constitute human attention abilities. Alertness is the ability to enter or maintain a state of alertness, and is measured through a reaction time to stimuli presented very briefly. Orientation is the ability to select information from sensory input and refers to the ability to focus attention on a particular space. Executive Function refers to the ability to inhibit and control distracting stimuli that conflict with target stimuli. The attentional control test includes four cue-related conditions: No Clue, Redundant Clue, Spatial Clue, and Central Clue. Also, the attentional control test includes three target stimuli-related conditions: Match, Mismatch, and Neutral. ‘No Clue’ refers to a condition in which no star-shaped clue is presented on the screen. ‘Double Clue’ refers to a condition in which star clues are presented at the top and bottom of the screen at the same time. ‘Spatial Clue’ refers to a condition in which a clue is presented only at the top or bottom of the screen. ‘Central Clue’ refers to a condition in which a clue is presented at the center of the screen. Among the target stimuli conditions, ‘Mismatch’ refers to a condition a condition where the middle arrow among five arrows presented on the screen points in a different direction. ‘Match’ refers to a condition where all the arrows point in the same direction. ‘Neutral’ refers to a condition where an arrow is presented at the center, but a horizontal bar appears on both sides of the arrow. A user is required to pay attention to the direction of the middle arrow and press a direction key indicated by the arrow. Of course, various cognitive function tests such as Spatial Span Task, Tower of London Task, Finger Tapping Test, and Balloon Analogue Risk Task (BART) may be used.

The deep sleep induction part 360 may monitor sleep stages through the user terminal 100 or a wearable device 400 linked to the user terminal 100, and induce brainwave entrainment using monaural beats or binaural beats preset for each sleep stage. At this point, it is possible to sleep induction and wake-up alarm may be employed to readjust a circadian rhythm of the user terminal 100, rapid sleep onset may be induced using brainwave entrainment of alpha and theta waves during sleep induction, deep sleep may be induced using brainwave entrainment of delta waves, and monoaural or binaural beats may be provided for brainwave entrainment of alpha, theta, and delta waves according to sleep stages. The theoretical basis for maintaining deep sleep and enhancing memory performance is supported by a study (Papalambros NA, Santostasi G, Malkani RG, Braun R, Weintraub S, Paller KA, Zee PC. Acoustic Enhancement of Sleep Slow Oscillations and Concomitant Memory Improvement in Older Adults. Front Hum Neurosci. 2017 Mar. 8;11:109. doi: 10.3389/fnhum.2017.00109. PMID: 28337134;PMCID: PMC5340797). Furthermore, The theoretical basis is also grounded in the paper that explored Alzheimer's disease patients entering deep sleep about an hour later than non-Alzheimer's cognitive impairment patients (Roh HW, Choi JG, Kim NR, Choe YS, Choi JW, Cho SM, Seo SW, Park B, Hong CH, Yoon D, Son SJ, Kim EY. Associations of rest-activity patterns with amyloid burden, medial temporal lobe atrophy, and cognitive impairment. EBioMedicine. 2020 Aug.;58:102881. doi: 10.1016/j.ebiom.2020.102881. Epub 2020 Jul. 28. PMID: 32736306; PMCID: PMC7394758).

The method for monitoring sleep stages is disclosed in another application by the same applicant of the present disclosure, but will be described briefly for the sake of understanding. Using the data collected from the wearable device 400, such as GPS, accelerometer, magnetometer, etc., it is possible to determine the current sleep stage of a user based on factors such as the user's movements (toss and turn), heart rate, and the like.

### Characteristics of Sleep Stages and Biological Signal Sensor

To accurately discern sleep states, it is essential to analyze the sleep stages within an entire sleep cycle. Generally, the human sleep cycle begins in the wake stage (WAKE) and predominantly consists of repeated stages of NREM and REM sleep. Physiological signals measured during sleep exhibit distinct characteristics across these stages. During the wake stage WAKE, muscles are activated, and breathing and heart rate are irregular. In the NREM stage, compared to WAKE, heart rate, breathing, and eye movements slow down. Upon entering the REM stage following NREM, the heart rate and breathing become rapid and irregular again, while muscle tone below the neck decreases.

### Feature Extraction

### Respiratory Rate

The respiratory rate is subject to noise due to a user's movements. Therefore, at certain points where the respiratory rate is 0 or the value is deemed too high or too low to be considered valid, the data can be corrected by averaging the values before and after. In the REM stage, breathing is characterized by irregularity. Considering the characteristic of increased amplitude due to irregular breathing, the maximum amplitude within a 3-minute window may be measured and used as a feature.

### Heart Rate

The heart rate stabilizes upon entering the NREM stage, and during the REM stage, the heart rate graph shows a trend of increasing and decreasing with larger amplitude compared to other sleep stages. Given these characteristics, the amplitude magnitude can be extracted using a 1-minute window.

### Electrocardiogram (ECG)

In ECG analysis, the Pan-Tompkins algorithm, which is commonly used, may be applied to extract the QRS complex. Three types of features may be extracted from ECG data: heart rate variability (HRV), amplitude, and the number of R-wave peaks within 1 minute. HRV analysis is based on detecting the R-peak, which is the most prominent feature of the QRS complex. HRV shows different characteristics for each sleep stage and may distinguish between arousal and sleep states with over 87% accuracy.

### Movement

Data collected from the 3-axis accelerometer is used to determine movement during sleep. Since directionality is not considered, the 3-dimensional accelerometer values may be reduced to a 1-dimensional value and converted into an intensity value that represents the intensity of movement during sleep.

### Sleep Cycle

Most living things have a 24-hour circadian rhythm. To assign a Clock Proxy value representing the sleep cycle instead of an absolute time corresponding to a measured biosignal, an existing biological clock modeling technique may be used to allocate values from the start to the end of the measurement period.

### Data Generation, Learning, and Classification

Raw data collected by each sensor may be visualized to interpret the waveforms of each sleep stage and then labeled as 0 for WAKE, 1 for NREM, and 2 for REM, based on comparisons with waveforms observed in previous studies. Processed data may then be used as training and test data for an SVM classifier. Each sleep stage may be classified using the SVM classifier.

### 90-Minute Golden Time Sleep Rule

According to Dr. Seiji Nishino, director of the Sleep and Circadian Neurobiology Research Institute at Stanford University, as introduced in the best-selling book “Stanford High-Efficiency Sleep Method,” the first sleep cycle immediately after falling asleep significantly influences the overall sleep quality, as shown in FIG. 4A. Within the first 70 to 90 minutes of the first sleep cycle, a non-REM sleep stage is observed. The non-REM sleep is a deep sleep stage that replenishes fatigue and consolidates memory. The 90 minutes during which the first non-REM sleep occurs is called the “golden time of sleep.” Sleeping deeply during the first 90 minutes after falling asleep may provide a refreshing feeling the next day, even with less total sleep time than usual. For modern individuals who inevitably have limited sleep time, achieving deep and stable sleep during the first cycle may be the best option. As wakefulness during the day increases, the desire to sleep also intensifies. This desire is called sleep pressure. The sleep pressure peaks during the first 90 minutes after falling asleep, that is, during the golden time of sleep. Sleeping deeply during the golden time of sleep significantly reduces sleep pressure, eases the urge to sleep, and diminishes fatigue. The golden time of sleep regulates the autonomic nervous system through sleep, promotes growth hormone secretion, and enhances brain function.

### Binaural Beats in the Inaudible Frequency Range

Brain waves may also be synchronized through monaural beats or binaural beats using inaudible frequencies. This is based on a study (Choi MH, Jung JJ, Kim KB, Kim YJ, Lee JH, Kim HS, Yi JH, Kang OR, Kang YT, Chung SC. Effect of binaural beat in the inaudible band on EEG (STROBE). Medicine (Baltimore). 2022 Jul. 1;101 (26): e29819. doi: 10.1097/MD.0000000000029819. PMID: 35777013; PMCID: PMC9239629.) that suggests that binaural beats with inaudible frequencies can induce specific brainwaves similar to the brainwave-inducing effects of binaural beats at typical audible frequencies.

Accordingly, brainwaves may be entrained using a monaural beats or binaural beats in an inaudible band. Just like the brainwave induction effects of binaural beats with audible frequencies, binaural beats in an inaudible band may also induce specific brainwaves. A theoretical basis for this may be grounded in the paper (Choi MH, Jung JJ, Kim KB, Kim YJ, Lee JH, Kim HS, Yi JH, Kang OR, Kang YT, Chung SC. Effect of binaural beats in the inaudible band on EEG (STROBE). Medicine (Baltimore). 2022 Jul. 1;101 (26): e29819. doi: 10.1097/MD.0000000000029819. PMID: 35777013; PMCID: PMC9239629.).

The wake-up alarm part 370 may output blue light to induce wakefulness before a preset alarm time, insert and output an alpha-band or beta-band flicker to induce wakefulness, and output monaural beats or binaural beats in an inaudible band for brainwave entrainment of alpha waves or beta waves to induce wakefulness. At this point, the theory behind blue light promoting arousal is based on studies such as Liu et al.'s study (Liu D, Li J, Wu J, Dai J, Chen X, Huang Y, Zhang S, Tian B, Mei W. Monochromatic Blue Light Activates Suprachiasmatic Nucleus Neuronal Activity and Promotes Arousal in Mice Under Sevoflurane Anesthesia. Front Neural Circuits. 2020 Aug. 18;14:55. doi: 10.3389/fncir.2020.00055. PMID: 32973462; PMCID: PMC7461971.) demonstrating blue light activating suprachiasmatic nucleus neuronal activity and promoting arousal in mice under sevoflurane anesthesia, and Figueiro and Leggett's study (Figueiro MG, Leggett S. Intermittent Light Exposures in Humans: A Case for Dual Entrainment in the Treatment of Alzheimer's Disease. Front Neurol. 2021 Mar. 9;12:625698. doi: 10.3389/fneur.2021.625698. PMID: 33767659; PMCID: PMC7985540.) showing delayed Dim Light Melatonin Onset (DLMO) when exposed to blue light during sleep.

The blue light therapy part 380 may output blue light for cognitive function enhancement. The blue light may be generated by at least one of an LED device, a band pass filter, a flashlight, and a display. The theoretical basis for the cognitive function enhancement by blue light is grounded in the paper indicating that light exposure mitigates cognitive decline (Figueiro MG, Leggett S. Intermittent Light Exposures in Humans: A Case for Dual Entrainment in the Treatment of Alzheimer's Disease. Front Neurol. 2021 Mar. 9;12:625698. doi: 10.3389/fneur.2021.625698. PMID: 33767659; PMCID: PMC7985540.) and the paper indicating that blue light exposure during the learning process improves cognitive performance equivalent to a 5-point increase in I.Q. (Lehrl S, Gerstmeyer K, Jacob JH, Frieling H, Henkel AW, Meyrer R, Wiltfang J, Kornhuber J, Bleich S. Blue light improves cognitive performance. J Neural Transm (Vienna). 2007;114(4):457-60. doi: 10.1007/s00702-006-0621-4. Epub 2007 Jan. 25. PMID: 17245536.). In this case, the wavelength of the blue light may be as shown in FIG. 4B, but is not limited thereto.

When outputting a breathing method to induce beta waves, which are brainwaves for awakening and improving concentration, the breathing method coaching part 390 may use blue light or light with a high color temperature, which suppresses melatonin and induces beta waves. On the other hand, when outputting a breathing method to induce gamma waves, which are brainwaves for enhancing memory, the breathing method coaching part 390 can use yellow or red light other than blue light. Here, the theoretical basis for red and yellow lights inducing melatonin is supported by the paper (Blume C, Garbazza C, Spitschan M. Effects of light on human circadian rhythms, sleep and mood. Somnologie (Berl). 2019 Sep.;23(3):147-156. doi: 10.1007/s11818-019-00215-x. Epub 2019 Aug. 20. PMID: 31534436; PMCID: PMC6751071) and FIG. 4C. During wakefulness, a situation opposite to the above-mentioned method may be induced. Furthermore, the theoretical basis that breathing patterns activate alpha and beta waves is grounded in the paper (Sinha M, Sinha R, Ghate J, Sarnik G. Impact of Altered

Breathing Patterns on Interaction of EEG and Heart Rate Variability. Ann Neurosci. 2020 Apr.;27 (2): 67-74. doi: 10.1177/0972753120950075. Epub 2020 Nov. 9. PMID: 33335359; PMCID: PMC7724429).

Furthermore, the theoretical basis for the fact that breathing regulates brain activity in the prefrontal cortex network, and that changes in breathing patterns are associated with variations in attention, wakefulness, and emotional states, is grounded in the paper (Basha D, Chauvette S, Sheroziya M, Timofeev I. Respiration organizes gamma synchrony in the prefronto-thalamic network. Sci Rep. 2023 May 26;13(1):8529. doi: 10.1038/s41598-023-35516-7. PMID: 37237017; PMCID: PMC10219931). Additionally, the theoretical basis for the fact that deep and slow breathing contributes significantly to maintaining cognitive function, enhancing attention, working memory, and spatial perception in the prevention of dementia is grounded in the paper (Lee SH, Park DS, Song CH. The Effect of Deep and Slow Breathing on Retention and Cognitive Function in the Elderly Population. Healthcare (Basel). 2023 Mar. 20;11(6):896. doi: 10.3390/healthcare11060896. PMID: 36981553; PMCID: PMC10047962). Furthermore, breathing exercises have a positive effect on cognitive function, and given the synchronization of neuronal activity with natural breathing, breathing activates the cortex, hippocampus, and amygdala, which are involved in memory performance, and this is grounded in the paper (Kang ES, Yook JS, Ha MS. Breathing Exercises for Improving Cognitive Function in Patients with Stroke. J Clin Med. 2022 May 20;11(10):2888. doi: 10.3390/jcm11102888. PMID: 35629013; PMCID: PMC9144753).

Furthermore, the fact that mindfulness training enhances executive cognition, sustained attention, and working memory is based on the paper by Chambers, R., Lo, B.C.Y., and Allen, N.B. (“The Impact of Intensive Mindfulness Training on Attentional Control, Cognitive Style, and Affect,” Cogn Ther Res 32, 303-322 (2008). https://doi.org/10.1007/s10608-007-9119-0), and also based on the paper showing that deep breathing practice performed immediately after learning a motor skill improved both short-term and long-term retention of the motor skill (Yadav, G., Mutha, P. Deep Breathing Practice Facilitates Retention of Newly Learned Motor Skills. Sci Rep 6, 37069 (2016). https://doi.org/10.1038/srep37069).

The memory management part 391 may insert and output a gamma-band or alpha-band flicker for brainwave entrainment of gamma waves or alpha waves for treatment of mild cognitive impairment and Alzheimer's disease, output monaural beats or binaural beats in an inaudible band for brainwave entrainment of gamma or alpha waves, and coach a breathing method to stimulate the parasympathetic nervous system and enhance alpha waves. According to iMediSync, the brain waves of people with mild cognitive impairment (FIG. 4D), Alzheimer's disease (FIG. 4E), and ADHD (FIG. 4F) are compared to normal people, and if 5 the brain waves can be made closer to those of normal people using brainwave entrainment, the above symptoms or conditions may be alleviated. Accordingly, if brainwave entrainment is used for mild cognitive impairment, Alzheimer's disease, ADHD, etc., these symptoms or diseases may be alleviated. In other words, each disease or symptom may be alleviated by entraining to compensate for excessive or insufficient brainwaves.

TABLE 5

| | | | | Light |
|---|---|---|---|---|
| Disorder | Light | Sound | Brainwaves | Therapy |
| Mild | Gamma | Monaural | Gamma waves | |
| Cognitive | Band/Alpha | Beats/Binaural | alpha waves | |
| Impairment | Band | Beats | | |
| ADHD | Beta Band | Monaural | Beta Waves | Blue Light |
| | | Beats/Binaural | | |
| | | Beats | | |

TABLE 6

| | | |
|---|---|---|
| FIG. 4D | Mild cognitive | In brainwaves of healthy elderly individuals, |
| & | impairment & | normal brainwaves like alpha waves observed |
| FIG. 4E | Alzheimer's | (yellow) |
| | disease | In brainwaves of mild cognitive impairment |
| | | patients, slow waves like theta waves observed |
| | | (green) |
| | | In brainwaves of Alzheimer's disease patients, |
| | | very slow waves like delta and theta waves |
| | | observed (blue) |
| FIG. 4F | ADHD | Pattern of excessive alpha waves + normal |
| | | alpha frequency in the frontal lobe (gray) |
| | | Pattern of excessive alpha waves + slowed |
| | | alpha frequency n the frontal lobe(yellow) |
| | | Pattern of excessive theta waves in the frontal |
| | | lobe (blue) |
| | | Pattern of excessive beta 3 waves (green) |

The human brain experiences gamma wave oscillations around 40 times per second when consciously perceiving an object or situation. According to multiple studies, individuals with mild cognitive impairment or Alzheimer's disease exhibit weakened gamma wave oscillations. Therefore, neuronal gamma synchronization may be utilized to enhance memory and cognitive functions. Various neurological disorders, including Alzheimer's disease, are known to have issues in this [gamma range]. Neuroscientist Annabel Singer, at the Georgia Institute of Technology and Emory University, stated that dementia patients show changes in gamma activity. Dr. Singer suggested that sensory stimulants, such as 40 Hz flickering light and sound, may synchronize deep brain neurons at the same frequency, potentially restoring lost connectivity. Experimental results in mice showed that sensory entrainment in gamma waves led to immune changes that remove beta-amyloid proteins, toxic peptides in Alzheimer's disease. The basis for this is grounded in the paper (Singer AC, Martorell AJ, Douglas JM, Abdurrob F, Attokaren MK, Tipton J, Mathys H, Adaikkan C, Tsai LH. Noninvasive 40-Hz light flicker to recruit microglia and reduce amyloid beta load. Nat Protoc. 2018 Aug.;13 (8): 1850-1868. doi: 10.1038/s41596-018-0021-x. PMID: 30072722.). The paper states that using 40 Hz white light (4,000 K) to drive 40 Hz neural activity activates microglia and reduces neurotoxic peptide amyloid beta in Alzheimer's disease (AD).

The intensive management part 393 may output blue light for brainwave entrainment of beta waves for treatment of ADHD, insert and output a beta-band flicker for brainwave entrainment of beta waves, and output monaural beats or binaural beats in an inaudible band for brainwave entrainment of beta waves. Here, a method for brainwaves and improvements for ADHD are described in Tables 5 and 6. ADHD is known to be associated with deficiencies in the neurotransmitters dopamine, serotonin, and norepinephrine. These imbalances have different effects on brain functioning, which are reflected in changes in brainwaves. Accordingly, the light of the beta band is output, and the theoretical basis for this is grounded in a study on the effects of LED lighting on human brainwaves (by Professor Jeong Gi-yeong from Seoul National University Hospital's Neurology Department and Seoul National University Hospital's Biomedical Research Institute). Furthermore, beta power and SMR power are indicators related to concentration, as evidenced by research showing that high color temperature lighting is effective for enhancing concentration. In addition, a predominant pattern of change observed in several disorders, including ADHD, schizophrenia, and OCD, involves an increase in power at lower frequencies (delta and theta) and a decrease in power at higher frequencies (alpha, beta, and gamma). This is based on the paper stating that the FDA approved the use of high theta and beta ratios as biomarkers for ADHD diagnosis (by Newson JJ, Thiagarajan TC. EEG Frequency Bands in Psychiatric Disorders: A Review of Resting State Studies. Front Hum Neurosci. 2019 Jan. 9;12:521. doi: 10.3389/fnhum.2018.00521. PMID: 30687041; PMCID: PMC6333694.).

The linkage part 395 may link the user terminal 100 to a wearable device 400 that measures at least one biometric information of a user.

Hereinafter, an operation process according to the configuration of the improved service providing server of FIG. 2 described above will be described in detail using FIGS. 3 and 4 as an example. However, it will be apparent that the embodiment is merely one of various embodiments of the present disclosure, and the present disclosure is not limited thereto.

Referring to FIG. 3A, (a) a server 300 for cognitive function enhancement operates a user terminal 100 to induce brainwave entrainment and provide light therapy for cognitive function enhancement. At this point, the server 300 may determine cognitive function enhancement and correlation from the user terminal 100 or wearable device 400 and then provide a result of the determination. Accordingly, it is possible to control feedback regarding how frequently, to what extent of intensity, and when to use light, sound, and blue light. In addition, the feedback may be used as data to understand which elements are more effective and which are not. Furthermore, it may be possible to induce motivation to use the service of the present disclosure more frequently or extensively when it is confirmed that the cognitive function has improved after using the service according to one embodiment of the present disclosure. In addition, cognitive function enhancement may be achieved through sleep induction as shown in (c) of FIG. 3A, wakefulness induction as shown in (d) of FIG. 3A, and breathing as shown in (a) of FIG. 3B. In addition, brainwave entrainment may be used to improve symptoms of mild cognitive impairment and Alzheimer's disease patients, as shown in (b) of FIG. 3B, and brainwave entrainment and light therapy may be used to improve ADHD patients' symptoms, as shown in (c) of FIG. 3B.

Any details not explained regarding the method for providing a cognitive function enhancement service using AI-based brainwave entrainment and light therapy, as illustrated in FIGS. 2 through 4, may be identical to those explained through FIG. 1 or may be easily inferred from the described content. Therefore, detailed descriptions thereof will be omitted.

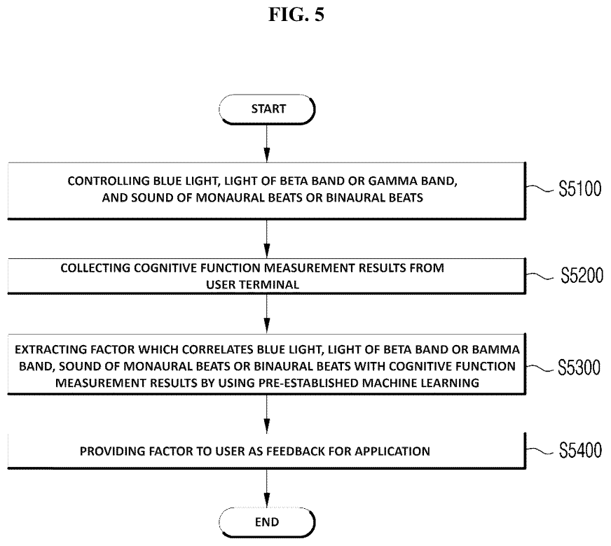

FIG. 5 is a diagram illustrating a process in which data is transmitted and received between components included in a system for providing a cognitive function enhancement service using AI-based brainwave entrainment and light therapy in FIG. 1 according to an embodiment of the present disclosure. Hereafter, an example of the process of data transmission and reception among the various components will be explained with reference to FIG. 5. However, it should be understood that the present disclosure is not limited to this embodiment, and it is evident to those skilled in the art that the process of data transmission and reception shown in FIG. 5 may vary according to the various embodiments previously described.

Referring to FIG. 5, a server for providing a cognitive function enhancement service controls blue light, light of beta band or gamma band, and sound of monaural beats or binaural beats in operation S5100.

In addition, the server then collects cognitive function measurement results from a user terminal in operation S5200 and, using pre-established machine learning, extracts a factor that correlates blue light, light of the beta band or gamma band, and sound of monaural or binaural beats with the cognitive function measurement results in operation S5300.

In addition, the server provides the factor to the user terminal as feedback for application in operation 5400.

The sequence between the above-described operations S5100 to S5400 is merely an example and is not limited thereto. That is, the sequence between the above-described operations S5100 to S5400 may change, and some of the operations may be executed simultaneously or omitted.

Matters that have not been explained about the method of providing a cognitive function enhancement service using AI-based brainwave entrainment and light therapy in FIG. 5 may be the same as those described with reference to FIGS. 1 to 4 or may be easily inferred from the descriptions of FIGS. 1 to 4. Therefore, those descriptions will be omitted.

A method of providing a cognitive function enhancement service using AI-based brainwave entrainment and light therapy according to an embodiment described in FIG. 5 may be implemented in the form of a recording medium containing computer-executable instructions or commands, such as an application or program module executable on a computer. The computer-readable medium may be an available medium that is accessible by a computer. The computer-readable storage medium may include a volatile medium, a non-volatile medium, a separable medium, and/or an inseparable medium. In addition, the computer-readable medium may include a computer storage medium. The computer storage medium may include a volatile medium, a non-volatile medium, a separable medium, and/or an inseparable medium that is implemented in any method or scheme to store computer-readable commands, data architecture, program modules, or other data or information.

The method of providing a cognitive function enhancement service using AI-based brainwave entrainment and light therapy according to an embodiment of the present disclosure may be executed by an application installed on a terminal, including a platform equipped in the terminal or a program included in the operating system of the terminal), or may be executed by an application (or program) installed by the user on a master terminal via an application provider server, such as a web server, associated with the service or method, an application, or an application store server. In this sense, the method of providing cognitive function enhancement services using AI-based brainwave entrainment and light therapy according to an embodiment of the present disclosure may be implemented in an application or program installed as default on the terminal or installed directly by the user and may be recorded in a recording medium or storage medium readable by a terminal or computer.

Although embodiments of the present disclosure have been described with reference to the accompanying drawings, it will be appreciated by one of ordinary skill in the art that the present disclosure may be implemented in other various specific forms without changing the essence or technical spirit of the present disclosure. Thus, it should be noted that the above-described embodiments are provided as examples and should not be interpreted as limiting. Each of the components may be separated into two or more units or modules to perform its function(s) or operation(s), and two or more of the components may be integrated into a single unit or module to perform their functions or operations.

It should be noted that the scope of the present disclosure is defined by the appended claims rather than the described description of the embodiments and include all modifications or changes made to the claims or equivalents of the claims.

What is claimed is:

1. A system for providing a cognitive function enhancement service using AI-based brainwave entrainment and light therapy, the system comprising:
a user terminal configured to output blue light for cognitive function enhancement, insert and output a beta-band or gamma-band flicker for brainwave entrainment of beta waves or gamma waves to enhance concentration and memory, output monaural or binaural beats in an inaudible band for brainwave entrainment to enhance concentration and memory, and collect cognitive function measurement results; and
a server for providing a cognitive function enhancement service, which comprises a controller configured to control the blue light, light of a beta band or gamma band, and sound of monaural or binaural beats; a collector configured to collect the cognitive function measurement results from the user terminal; an extractor configured to extract a factor that correlates the blue light, the light of the beta band or gamma band, and the sound of the monaural or binaural beats with the cognitive function measurement results by using pre-established machine learning; and an application part configured to provide the extracted factor to the user terminal for application.

2. The system of claim 1, wherein the server further comprises a task part configured to provide an attention network task based on Attention Networks Theory to obtain the cognitive function measurement results.

3. The system of claim 1,
wherein the blue light is generated by at least one of an LED device, a bandpass filter, a flashlight, and a display, and
wherein the light of the beta band or gamma band is controlled to generate a flicker by inserting image frames with flickering effects into video content, adjusting a display refresh rate, or flashing at least one of the display, the flashlight, and the LED device.

4. The system of claim 1,
wherein the server further comprises a deep sleep induction part configured to monitor sleep stages through the user terminal or a wearable device linked to the user terminal, and induce brainwave entrainment using monaural beats or binaural beats preset for each sleep stage,
wherein sleep induction and wake-up alarm are used to readjust a circadian rhythm of the user terminal,
wherein rapid sleep onset is induced using brainwave entrainment of alpha and theta waves during sleep induction, and deep sleep may be induced using brainwave entrainment of delta waves, and
wherein monoaural or binaural beats are provided for brainwave entrainment of alpha, theta, and delta waves according to sleep stages.

5. The system of claim 1, wherein the server further comprises a wake-up alarm part configured to output blue light to induce wakefulness before a preset alarm time, insert and output an alpha-band or beta-band flicker to induce wakefulness, and output monaural beats or binaural beats in an inaudible band for brainwave entrainment of alpha waves or beta waves to induce wakefulness.

6. The system of claim 1,
wherein the server further comprises a blue light therapy part configured to output blue light for cognitive function enhancement, and
wherein the blue light is generated by at least one of an LED device, a bandpass filter, a flashlight, and a display.

7. The system of claim 1, wherein the server further comprises a breathing coaching part configured to, use blue light or light with a high color temperature, which suppresses melatonin and induces beta waves when outputting a breathing method to induce beta waves, which are brainwaves for awakening and improving concentration, and to use yellow or red light other than blue light when outputting a breathing method to induce gamma waves, which are brainwaves for enhancing memory.

8. The system of claim 1, wherein the server further comprises a memory management part configured to insert and output a gamma-band or alpha-band flicker for brainwave entrainment of gamma waves or alpha waves for treatment of mild cognitive impairment and Alzheimer's disease, output monaural beats or binaural beats in an inaudible band for brainwave entrainment of gamma or alpha waves, and coach a breathing method to stimulate the parasympathetic nervous system and enhance alpha waves.

9. The system of claim 1, wherein the server further comprises an intensive management part configured to output blue light for brainwave entrainment of beta waves for treatment of ADHD, insert and output a beta-band flicker for brainwave entrainment of beta waves, and output monaural beats or binaural beats in an inaudible band for brainwave entrainment of beta waves.

10. The system of claim 1, wherein the server further comprises a linkage part configured to link the user terminal to a wearable device that measures at least one biometric information of a user.
